(19) United States
(12) Patent Application Publication
(10) Pub. No.: US 2003/0166568 A1
(43) Pub. Date: Sep. 4, 2003

(54) Organic compounds

(76) Inventors: Franz Kricek, Biedermannsdorf (AT); Andreas Nechansky, Wien (AT); Michael Robertson, San Diego, CA (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/221,984

(22) PCT Filed: Mar. 22, 2001
(86) PCT No.: PCT/EP01/03267

(30) Foreign Application Priority Data
Mar. 23, 2000 (US) 60191670

(51) Int. Cl. A61K 38/10; A61K 38/08; G01N 33/53

(52) U.S. Cl. 514/14; 514/16; 514/17; 435/7.1; 514/15

(57) ABSTRACT

Screening assay and methods comprising peptide PREKY for the identification of ligands, and ligands identified by such assay, which interfere with cell activation induced via FcεRIα, e.g. which inhibit mast cell/basophil activation.

The invention relates to compounds inhibiting mast cell/basophil activation. It concerns a method for the identification of ligands that interfere with cell activation induced via FcεRIα, comprising the peptide amino acid sequence PREKY as a molecular target.

Allergic response in connection with e.g. atopic diseases involves interaction of human IgE with its high affinity receptor FcεRI, e.g. expressed on the surface of mast cells and basophils. FcεRI is a member of the immunoglobulin (Ig) superfamily and binds human IgE with high affinity (KD=10⁻¹⁰M). FcεRI may e.g. be expressed either as a tetrameric (αβγ₂) multi-subunit complex in mast cells and basophils or as a trimeric (αγ₂) complex on monocytes, eosinophils, Langerhans and dendritic cells. The FcεRIα-subunit is an integral membrane protein with an extracellular region ecFcεRIα comprising two Ig-like domains, i.e. α1, membrane distal and α2 membrane proximal, which contain the complete high affinity IgE binding site. Allergen cross-linning of FcεRI-bound IgE initiates receptor aggregation and subsequent cellular activation results in the release of vasoactive and bronchoconstrictive mediators which are either preformed, e.g. histamine, or newly generated, like e.g. leukotrienes. These substances trigger cell activation, e.g. leading to the clinical symptoms of type I immediate hypersensitivity.

Crosslinking of receptor can also be accomplished IgE-independently, e.g. by antibodies directed against the extracellular part of the high affinity IgE receptor a-subunit (ecFcεRIα). Such anti-FcεRIα autoantibodies may cause at least part of the symptoms in chronic urticaria. Anti-ecFcεRIα monoclonal antibody (mAb) 5H5F8, which belongs to the IgG/K subtype, was raised against a fission protein consisting of a k-light chain fused to the amino terminus of recombiant ecFcεRIα(Val¹-Leu¹⁷⁹)(numbering according to Shimizu et al., PNAS 85 [1988] 1907-1911) and was characterized by Nechansky et al., Hybridoma 16 (1997) 441-446. 5H5F8 binds to immobilized ecFcεRIα in enzyme-linked immunosorbent assays but does not inhibit IgE binding to immobilized ecFcεRIα. Furthermore, using a bacterially displayed random peptide library the 5H5F8 epitope was mapped to the ecFcεRIα amino acid stretch P¹⁷³REKY¹⁷⁷ (Nechansky et al., FEBS L. 441 [1998] 225-230), which lies close to the cell membrane.

It has now been found that 5H5F8 is not anaphylactogenic when tested on human peripheral blood leukocytes (hPBLs) and that 5H5F8 does inhibit allergen-induced sulfidoleukotriene release from IgE-sensitized human pheripherial blood leukocytes—but not by preventing IgE binding to ecFcεRIα. It was further found that chemically synthesized peptides comprising the 5H5F8 epitope (PREKY) are recognized by 5H5F8.

According to the present invention the peptide amino acid sequence PREKY may be used in a method for the identification of ligands that interfere with cell activation induced via FcεRIα, e.g. as a molecular target in a screening assay. A compound (ligand) which binds to the amino acid sequence PREKY may—in homology to mAb 5H5F8—inhibit allergen-induced sulfidoleukotriene release from IgE-sensitized human peripheral blood leukocytes. As a consequence, a so-identified compound may interfere with the signal transduction cascade responsible for cell activation after crosslinkage of FcεRIα—bound human IgE. Compounds identified by such a screening assay, e.g. in the form of a pharmaceutical composition comprising an effective amount of such a ligand in combination with pharmaceutically acceptable excipients or carriers or diluents, may therefore be useful in the inhibition of mast cell/basophil activation involved in allergic response, which is connected with e.g. atopic diseases. A ligand identified according to the present invention may thus be useful in the treatment of atopic diseases, such as allergic asthma (AA), allergic rhinitis (AR), atopic dermatitis (AD) and chronic urticaria (CU) mediated by auto-antibodies directed against IgE or FcεRIα.

In one aspect the present invention provides a screening assay for the identification of a ligand (which is e.g. useful in the inhibition of mast cell/basophil activation) that interferes with cell activation induced via FcεRIα, comprising the peptide amino acid sequence PREKY as a molecular target.

“Peptide amino acid sequence PREKY” as used herein includes the peptide having the amino acid sequence PREKY (Seq. id. no. 1) and any peptide or polypeptide containing the amino acid sequence of the peptide PREKY, e.g. as a part of the whole sequence, e.g. a peptide, e.g. of the amino acid sequence NITVIKAPREKY (Seq. id. no. 2), ESEPLNITVIKAPREKYWL (Seq. id. no. 3) or KAPREKYWL (Seq. id. no. 4).

“Ligand” as used herein define any compound which binds to peptide amino acid sequence PREKY.

A screening assay may be e.g. as follows: PREKY or chemically synthesized (poly)peptides comprising the amino acid sequence PREKY are immobilized onto a surface, e.g. a plastic surface, creating a selection matrix for potential ligands. Candidate compounds comprising possible ligands may be provided as appropriate, e.g. by a method as conventional, e.g by provision of libraries, e.g. phage libraries which display random peptides of various lengths (Devlin et al, Science 249 [1990] 404-406; Cortese et al., Curr. Opin. Biotech. 6 [1995] 73-80) or random peptide libraries displayed on the flagella of Escherichia coli (Lu et al., Biotechnology 13 [1995] 366-372). After enrichment of specifically binding clones by several rounds of panning against the selection matrix, binding clones are isolated and the amino acid sequence of the peptides displayed by these clones are determined by translation of the encoding DNA sequence. The identified amino acid sequences may serve as templates for the synthesis of peptides which can be tested e.g.

a) for binding to FcεRIα; and

b) for their capability of inhibiting allergen induced mediator release from IgE sensitized cells.

Test methods which ascertain the binding of a ligand to FcεRIα or the capability of a peptide which inhibits allergen-induced mediator release from IgE-sensitized cells are known or may be carried out as appropriate, e.g. as described herein (Example 2).

Other ligands may comprise combinatorial libraries e.g. immobilized on beads. Beads that contain substances that bind to peptide amino acid sequence PREKY which is immobilized onto a surface can be identified e.g. by confocal nanomicroscopy, which is an established and well-known method. The bead can be picked specifically and the immobilized compound can be cleaved off and identified, e.g. by nuclear magnetic resonance analysis.

In another aspect the invention provides a method of identifying a ligand to peptide amino acid sequence PREKY comprising

contacting a candidate compound with a molecular target comprising peptide amino acid sequence PREKY,

determining if said candidate compound is bound to peptide amino acid sequence PREKY,

isolating the ligand bound to peptide amino acid sequence PREKY, and

identifying the candidate compound.

The invention further comprises a ligand identified by a method or screening assay as defined above.

A ligand identified by said method or screening assay includes e.g. a peptide, a peptidomiinetic, an antibody, a fragment of an antibody or a chemical entity, e.g. a LMW (low molecular weight) compound.

A ligand identified by a method or screening assay as described above may be useful in

inhibition of allergen-induced sulfidoleukotriene release from IgE-sensitized human pheripherial blood leukocytes,

interfering with the signal transduction cascade responsible for cell activation after crosslinkage of FcεRIα bound human IgE,

inhibition of mast celylbasophil activation; and

treatment of atopic diseases, such as allergic asthma (AA), allergic rhinitis (AR), atopic dermatitis (AD) and chronic urticaria (CU) mediated by auto-antibodies directed against IgE or FcεRIα.

In another aspect the invention provides the use of a ligand identified by the method above as a pharmaceutical, e.g. in the treatment of atopic diseases, such as allergic asthma (AA), allergic rhinitis (AR), atopic dermatitis (AD) and chronic urticaria (CU) mediated by auto-antibodies directed against IgE or FcεRIα.

In another aspect the invention provides a pharmaceutical composition comprising an effective amount of a ligand identified by a method or screening assay as described above in combination with pharmaceutically acceptable excipients or carriers or diluents.

The invention also comprises a method of treatment by inhibition of mast cell/basophil activation, comprising administering a pharmaceutically effective amount of a ligand identified by a method or screening assay as described above to a subject, e.g. a mammal in need of such treatment.

In another aspect the present invention provides a method for the inhibition of mast cell/basophil activation comprising administering a pharmaceutically effective amount of a ligand identified by a method or screening assay as described above, e.g. in the form of a pharmaceutical composition comprising a ligand identified by a method or screening assay as described above, to a subject in need of such treatment.

In another aspect the present invention provides a method for ident gilmg an FcεRIαligand that interferes with allergen-induced and FcεRIα-mediated cell activation, comprising providing a selection matrix comprising peptide amino acid sequence PREKY in a fluid environment in the presence of a candidate compound, determining whether said candidate compound binds to said selection matrix, identifying a candidate compound which binds to said selection matrix, and isolating the ligand which binds to said selection matrix.

A candidate compound which binds to the selection matrix is considered to be a potential inhibitor of FcεRIα mediated cell activation. Such a compound may be (dependent on the library used for screening) e.g a peptide, a peptidomirinetic, an antibody or a fragment of an antibody.

### EXPLANATION OF THE FIGURES

FIG. 1 shows that 5H5F8 is not anaphylactogenic when tested on hPBL's. The anaphylactogenic potential of 5H5F8 is determined in normal peripheral blood leukocytes using antibody-triggered release of sLT. As appears from FIG. 1, 5H5F8 and two further anti-human FcεRIα mAbs (15/1 and 6F9G9) induce no sLT release. The CAST-ELISA kit positive control mAb, which binds within FcεRIα, crosslinks adjacent receptors resulting in release of sLT to essentially the same extent as cells incubated with JW8 IgE and subsequently triggered with NIP-BSA. Incubation of cells with anti-human IgE results in sLT release to about the same extent as in case of use of pc. anti-FcεRIα, indicating that the primary PBLs are at least partially loaded with endogenous IgE. Details are as set out in Example 1.

FIG. 2 shows that 5H5F8 inhibits antigen-specific cell trigger of IgE-sensitized hPBLs. Co-incubation of JW8 IgE with the maximal amount of mAb 5H5F8 results in a significant reduction (ca. 50%) of sLT release after NIP-BSA trigger. No inhibition is observed using an isotype-matched control (anti-IL8) antibody. 5H5F8 treatment only (50 μg/ml) has no direct effect on sLT release. For comparison the inhibitory effect of mAB 15/1 is determined which blocks the binding site for IgE on FcεRIα. A concentration-dependent inhibition of sLT release in a concentration of ca. 5 μg/ml is found. Because of the possibility that whole 5H5F8 might bind simultaneously and cross-link human FcεRIα via the Fab portion and FcγRIIb via the mAb portion, leading to down-regulation of cellular activation, the 5H5F8 Fab fragment is tested for its capacity to inhibit sLT release. Treatment with the highest concentration of Fab affords ca. 40% reduction in sLT release, confirming the specificity of this mAb for FcεRIα and excluding the possibility of 5H5F8 -mediated activity derived from FcεRI-FcγRIIb ligation. Details are as set out in Example 2.

FIG. 3 shows peptide sequences identified by screening the FliTrx random peptide library with 5H5/F8. Details are as set out in Example 3.

FIG. 4 shows that peptides containing the amino acid sequence PREKY are recognized by 5H5F8. Details are as set out in Example 4.

The following Examples illustrate the invention. All temperatures are in degrees Centigrade.

### EXAMPLE 1

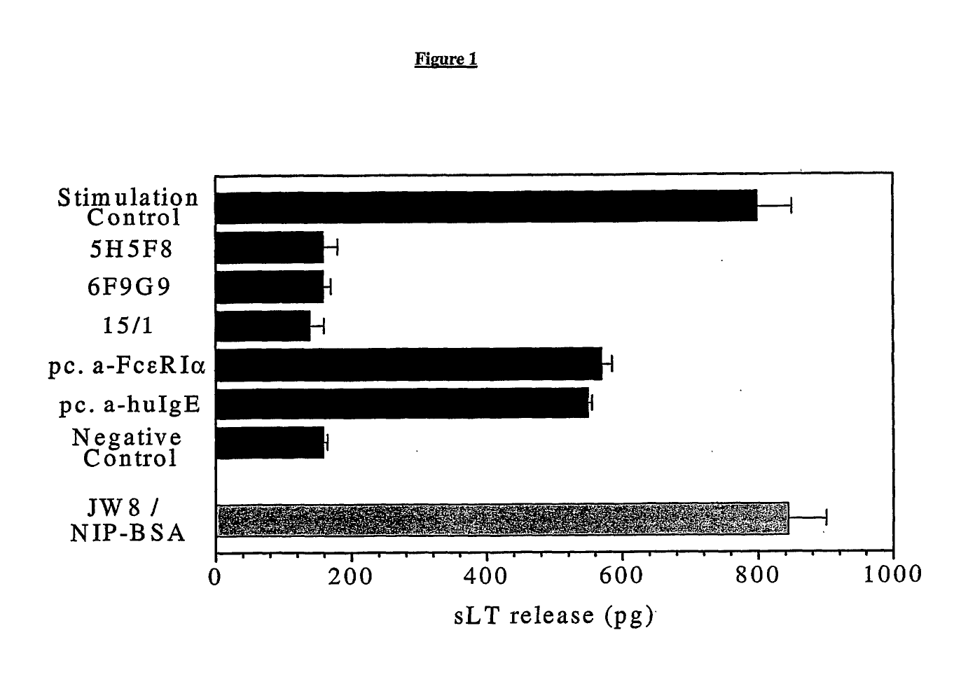

Isolation of human peripheral blood leukocytes (hPBLs) and sulfidoleukotriene (sLT) quantitation is performed using the CAST-ELISA (Bühlmann Laboratories AG, Allschwil, Switzerland) according to the manufacturer's protocol with mAbs 6F9G9 and 5H5F8 and polyclonal (pc.) sera pc. a-huIgE and pc. a-FcεRIα at the concentration indicated in the protocol. For sensitization the human chimeric IgE (transfectoma JW8/5/13; ECACC, Porton Down, UK, ref. No. 87080706) designated JW8 and consisting of mouse V_{H} and human C_{ε}chains and specific for the hapten 4-hydroxy-3-nitrophenylacetyl (NIP) is used. Release of sLT from JW8 IgE-sensitized hPBLs is determined in cell supernatants after trigger with NIP conjugated to bovine serum albumin (BSA) designated NIP-BSA. PBLs are isolated from whole blood (3ml)-after addition of dextran solution (750 μl) and incubation at 25° for 90 minutes. 1400 μl of the upper phase are sedimented and the cell pellet is resuspended in 850 μl of stimulation buffer. 200 μl of the cell suspension obtained are added to tubes containing either 1.25 μg of JW8 IgE, or 1.25 μg of mAb 6F9G9, or 1.25 μg of mAb 5H5F8, each in 50 μl of interleukin-3 containing stimulation buffer (CAST-ELISA kit component). Sensitization is performed at 37° for 50 minutes with gentle mixing after 25 minutes. JW8 sensitized cells are triggered with 100 ng/ml of NIP-BSA for 40 minutes at 37°. Released sLT is quantitated by competition ELISA using a constant amount of alkaline phosphatase-labeled sLT (sLT-AP) to compete with cellular sLT. After the last incubation samples are briefly centrifuged (1000 g) and 200 μl supernatant aliquots are mixed with 200 μl of a 1:1 mixture of sLT-AP solution and ELISA buffer. Anti-mouse IgG precoated microtiter plates are incubated with 50 μl of the mouse anti-sLT mAb (kit component) at 25° for 2 h. Wells are washed twice with phosphate buffered saline (PBS, 150 mM NaC1, 3 mM KCI, 8 mM Na₂HPO₄, 1.5 mM K₂PO₄, pH≈7.5) and 195 μl of the sLT containing sample is added in duplicates and allowed to incubate for 90 minutes at 25°. Wells are washed twice with 300 μl of PBS and developed with p-dinitrophenylphosphate substrate for 40 min at 25°. The OD at 405 nm is recorded and the amount of secreted sLT determined according to the CAST-ELISA evaluation protocol. Results are set out in FIG. 1. From FIG. 1 it is evident that human PBLs are not activated by 5H5F8 and can be triggered in an antigen-specific way by NIP-BSA after sensitization with JW8 IgE. MAbs and antisera are used at a concentration of 100 μg/ml. As a stimulation control a monoclonal anti-FcεRIα antibody is used to cross-link surface FcεRI. The negative control is carried out by determination of non-specific release of sLT using buffer only and the anti-FcεRIα mAb 6F9G9 is used as IgG1/k isotype control. A rabbit polyclonal anti-FcεRIα antiserum (pc. a-FcεRIα) and a goat pc. anti-human IgE antiserum (pc. a-huIgE) are also included as positive controls. In addition, NIP-BSA specific trigger (100 ng/ml) of JW8 IgE sensitized (2 μg/ml) cells is shown. Pg represents mean values of duplicate samples determined from 100 μl cell aliquots.

### EXAMPLE 2

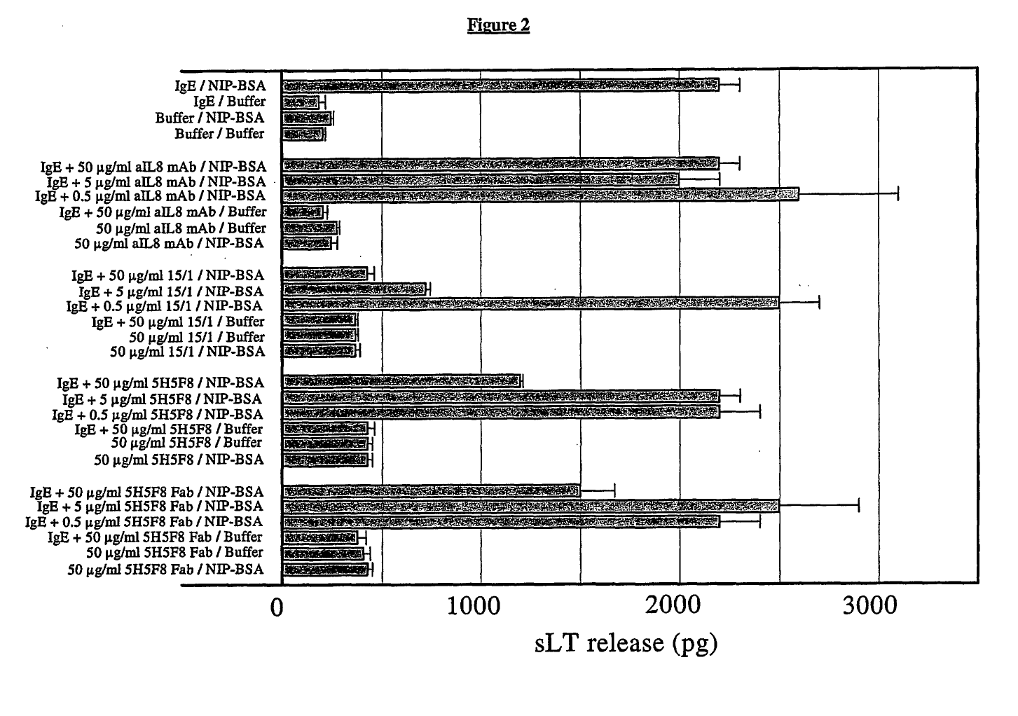

Isolation of human peripheral blood leukocytes (bPBLs) and sulfidoleukotriene (sLT) quantitation is performed with the CAST-ELISA (Büuhlmann Laboratories AG; Allschwil, Switzerland) according to the manufacturer's protocol with the mAbs aIL8, 5H5F8 and 5H5F8 Fab and polyclonal (pc.),rabbit anti-human ec FcεRIα serum and pc. goat anti-human IgE serum (purchased from Nordic) used at the concentration indicated for each experiment. Release of sLT from JW8 IgE-sensitized hPBLs is determined in cell supernatants after NIP-BSA trigger. As a control monoclonal antibody the anti-FcεRIα mAb 15/1 is used which blocks IgE binding to FcεIα. PBLs are isolated from whole blood (3 ml) after addition of dextran solution (750 μl) and incubation at 250 for 90 minutes. 1400 μl of the upper phase are sedimented and the cell pellet is resuspended in 850 μl of stimulation buffer. 200 μl of the cell suspension obtained are added to tubes each containing a 50 μl mixture containing either JW8 IgE (5 μg/ml) alone or in combination with either the mAb aIL8, or 5H5F8, or 5H5F8 Fab, in concentrations ranging from 0.5 μg/ml to 50 μg/ml. The mixture in a tube is incubated for 15 hours at 4° with gentle mixing. The cells are washed and triggered with NIP-BSA (100 ng/ml) for 40 minutes at 37° and assayed for secreted sLT by CAST ELISA Secreted sLT are quantitated as described in Example 1. Sulfidoleukotriene release is shown in picogram (pg) and represents mean values of duplicate assays of sLT release from 100 μl cell aliquots. Results are set out in FIG. 2.

As shown in FIG. 2, 5H5F8 inhibits antigen-specific NIP-BSA cell trigger of IgE-sensitized hPBLs.

### EXAMPLE 3

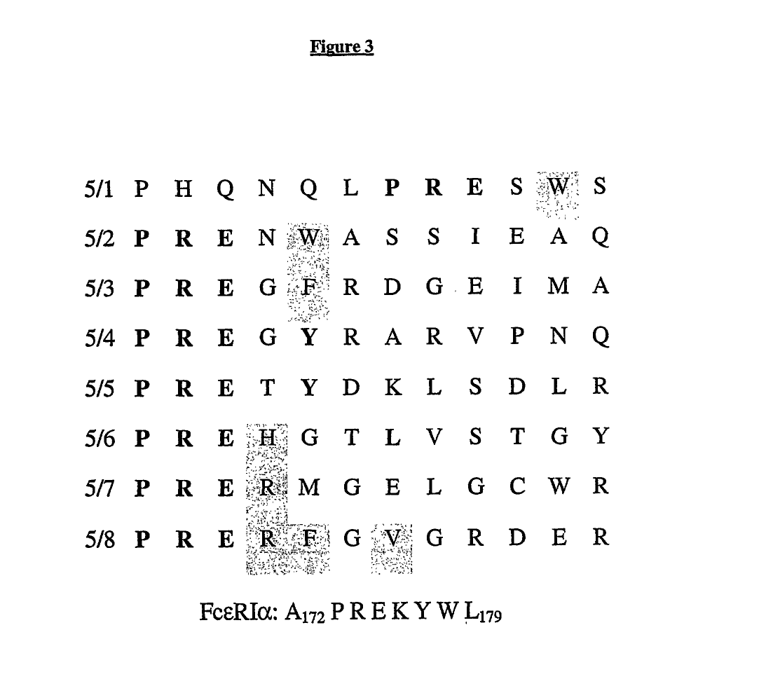

The epitope is identified using the pFliTrx random peptide library (Invitrogen, San Diego, Calif., USA). This random dodecapeptide library is expressed on the flagella of Escherichia coli bacteria after induction with tryptophan. The panning procedure and colony blot analysis is performed following the protocol of Lu et al., Biotechnology 13 [1995 ] 366-372). Bacterial clones being positive regarding 5H5F8 binding in dot blot experiments are further analysed in Western blot experiments under non reducing conditions. 8 positve clones are selected and cultured overnight in 1.5 ml LB medium containing 100 μg/ml of ampicillin. Flagella expression is induced by addition of 100 μg/ml of tryptophan. Cultures are spun down, the pellet obtained is redissolved in 200 μl SDS sample buffer and boiled for 5 minutes. 10 μl of the sample is applied to a 1 mm 14% Tris-glycine minigel (Novex, San Diego, Calif., USA) in 1.25% SDS and separated for 90 minutes at constant voltage of 125 volts. The separated proteins are transferred to a 0.2 μm polyvinylidene difluride (PVDF) membrane (Novex). The membrane is blocked with 5% non-fat dry milk (Biorad) in TBS (20 mM Tris, 500 mM NaC1 pH 7.5) for 1 hour and washed twice for 10 minutes with TBST (0.05% Tween 20-TBS, pH 7.5). The membrane obtained is incubated with 1% non-fat dry milk (Biorad) in 20 ml TBS containing 3 μg/ml of 5H5F8 for 1 hour at room temperature. The membrane is washed twice for 10 minutes with TBS_{T} and incubated with a goat anti-mouse IgG HRP conjugate (BioRad) for 1 hour at room temperature. After washing four times for 10 minutes with TBS_{T} the blots are developed using the ECL substrate (Amersham, instruction protocol) and exposed to ECL hyperfilm (Amersham) for 1 minute. To test for flagella expression, blots are submerged in stripping buffer (100 mM 2-ME, 2% SDS and 62.5 mM Tris-HCl, pH 6.7) and incubated at 50° for 45 minutes. The membranes obtained are washed thrice with TBST, blocked, and reprobed with a 1:5000 dilution of an anti-thioredoxin mAb (Invitrogen) following the procedure described above. 8 binding clones (designated 5/1 to 5/8) are selected and the sequence of each insert encoding for the displayed peptide is determined by DNA sequencing. After translation, a consensus sequence is obtained which can be aligned to the FcεRIα region Pro¹⁷³ to Tyr¹⁷⁷. The amino acid sequences of the dodecameric peptides are shown in FIG. 3 in the single letter code. Shaded amino acids belong to a consensus sequence that can be aligned by similar amino acid properties to the ecFcεRIα sequence whereas amino acids typed bold represent identities.

### EXAMPLE 4

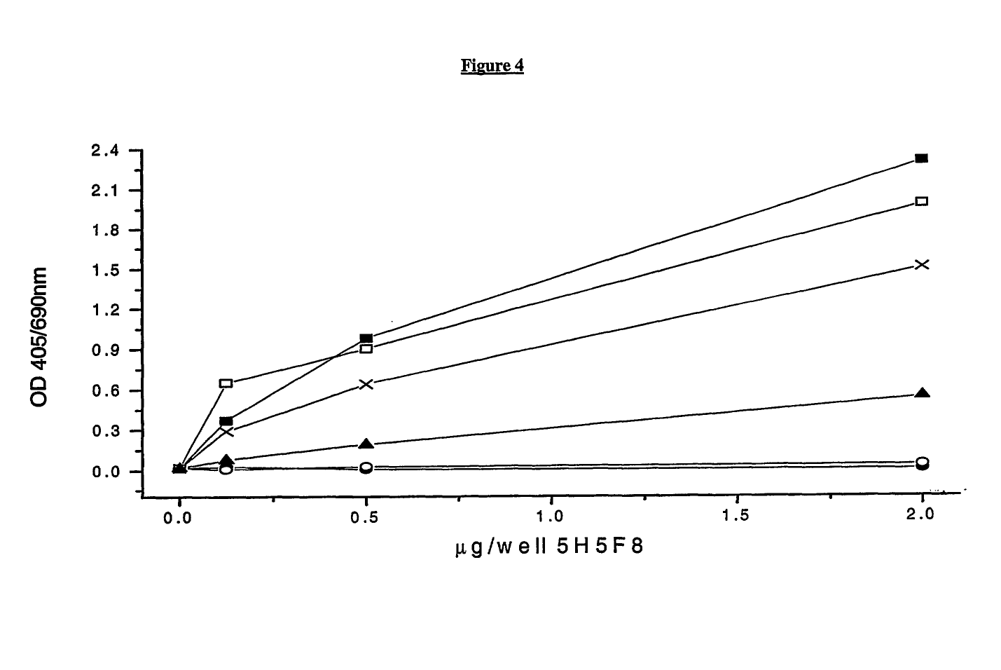

1 mg/ml of the linear FcεRIα-derived synthetic peptides ESEPLNITVIKAPREKYWL (Seq. id. no. 3), ESEPLNITVIKAPRE (Seq. id. no. 5), NITVIKAPREKY (Seq. id. no. 2), KAPREKYWL (Seq. id. no. 4), PREKY (Seq. id. no. 1) and NITVI (Seq. id. no. 6), all more than 80% pure (HPLC) and characterized by mass spectroscopy are each dissolved in coupling buffer (0.1M NaHCO₃, 0.5M NaCl, pH: 8.5, NaOH) and stored at −20°. For coupling, solutions are diluted 1:50 with coupling buffer and 100 μl dispensed to each well of a Labcoat Amine Binding Plate (Costar) and incubated for 12-14 h at 4° in a humidified chamber. Peptides immobilized on the plates are obtained. Remaining binding sites of the immobilized peptides are blocked after washing the wells obtained thrice with PBS containing 0.05% Tween-20, pH 7.4 (PBS_{T}) by treatment with 200 μl of blocking buffer (0.2 M glycine, 1% BSA, 0.05% Tween-20 in PBS, pH 8.5) for 3 h at 25°. Plates obtained are washed and amounts of 5H5F8 as specified in FIG. 4 are added, followed by incubation for 2 hours at 25°. After washing, 100 μl/well of a 1:1000 diluted goat anti-mouse IgG-HRP conjugate (BioRad) is dispensed and incubated for 90 min at 25°. Wells are washed twice and then incubated with 100 μl per well of ABTS substrate solution (BioRad). The reaction is stopped by addition of 100 μl 3% oxalic acid and the absorbance is read at 405 nm. Non-specific binding is assessed by mAb binding to mock-coupled (peptide-free) wells.

Results are set out in FIG. 4. In FIG. 4 values obtained by use of NITVI are indicated by the sign -●-, values obtained by use of ESEPLNITVIKAPRE are indicated by the sign -∘-, values obtained by use of ESEPLNITVIKAPRE are indicated by the sign -x-, values obtained by use of NITVIKPREKY are indicated by the sign -▪-, values obtained by use of KAPREKYWL are indicated by the sign -□- and values obtained by use of PREKY are indicated by the sign -♦-. O.D values indicated in FIG. 4 are corrected by subtraction of background binding to wells without peptide and represent mean values of duplicates. From FIG. 4 it is evident that 5H5F8 binds to PREKY and PREKY containing peptides (or protein) (and does not bind in case of peptides which do not contain PREKY).

1. A screening assay for the identification of a ligand that interferes with cell activation induced via FcεRIα, comprising peptide amino acid sequence PREKY as a molecular target.

2. A screening assay according to claim 1 in the identification of a ligand which is useful in the inhibition of mast cell/basophil activation.

3. A method of identifying a ligand to peptide amino acid sequence PREKY comprising
contacting a candidate compound with a molecular target comprising peptide amino acid sequence PREKKY,
determining if said candidate compound is bound to peptide amino acid sequence PREKY,
isolating the ligand bound to peptide amino acid sequence PREKY, and
identifying the candidate compound.

4. A method for identifying an FcεRIα ligand that interferes with allergen-induced and FcεRIα-mediated cell activation, comprising providing a selection matrix comprising peptide amino acid sequence PREKY in a fluid environment in the presence of a candidate compound, determining whether said candidate compound binds to said selection matrix, identifying a candidate compound which binds to said selection matrix, and isolating the ligand which binds to said selection matrix.

5. A screening assay according to claim 1 or 2 or a method according to claim 3 or 4 wherein the peptide amino acid sequence PREKY is selected from PREKY (Seq. id. no. 1), ESEPLNITVIKAPREKYWL (Seq. id. no. 3), NITVIKAPREKY (Seq. id. no. 2) and KAPREKYWL (Seq. id. no. 4).

6. A ligand identified by a method or a screening assay according to any one of claims 1 to 4.

7. Use of a ligand identified by a method or a screening assay according to any one of claims 1 to 4 as a pharmaceutical.

8. A pharmaceutical composition comprising an effective amount of a ligand identified by a method or a screening assay according to any one of claims 1 to 4 in combination with at least one pharmaceutically acceptable excipient or carrier or diluent.

9. A method of treatment by inhibition of mast cell/basophil activation comprising administering a pharmaceutically effective amount of a ligand identified by a method or a screening assay according to any one of claims 1 to 4 to a subject in need of such treatment.

10. A ligand identified by a method or a screening assay according to any one of claims 1 to 4 which is a peptide, a peptidomimetic, an antibody, a fragment of an antibody or a chemical entity.
